(19) United States
(12) Patent Application Publication
(10) Pub. No.: US 2023/0414152 A1
(43) Pub. Date: Dec. 28, 2023

(54) MINIMIZATION OF ECG INTERFERENCE IN PATIENT MONITORING SYSTEMS

(71) Applicant: DRAGERWERK AG & CO, KGAA, Lubeck (DE)

(72) Inventors: LIXIN LIU, BROOKLINE, MA (US); DEREK Y. QIN, ANDOVER, MA (US)

(21) Appl. No.: 18/213,188

(22) Filed: Jun. 22, 2023

Related U.S. Application Data
(60) Provisional application No. 63/355,862, filed on Jun. 27, 2022.

(51) Int. Cl. A61B 5/339; A61B 5/355

(52) U.S. Cl. CPC A61B 5/339; A61B 5/355; A61B 5/303

(57) ABSTRACT

A patient monitoring system includes a patient monitor and an ECG lead set coupled thereto. The patient monitor may be programmed to periodically issue interrogation signals to the ECG lead set to determine the type and connection status of the ECG lead set. The interrogation signals may be issued via a communication bus. The patient monitor may time the issuance of interrogation signals to the ECG lead set to correspond to non-critical ECG intervals, such that interference with the ECG signals by the communication bus is minimized.

### CROSS-REFERENCE TO RELATED APPLICATIONS

The priority and earlier effective filing date of U.S. Application Ser. No. 63/355,862, filed Jun. 27, 2022, is hereby claimed for all purposes, including the right of priority. This related application is also hereby incorporated by reference for all purposes as if expressly set forth verbatim herein.

### TECHNICAL FIELD

The present disclosure pertains to electrocardiogram (“ECG”) monitoring, and more particularly to minimization of interference with ECG signals received by a patient monitor.

### DESCRIPTION OF THE RELATED ART

This section of this document introduces information about and/or from the art that may provide context for or be related to the subject matter described herein and/or claimed below. It provides background information to facilitate a better understanding of the various aspects of the that which is claimed below. This is a discussion of “related” art. That such art is related in no way implies that it is also “prior” art. The related art may or may not be prior art. The discussion in this section of this document is to be read in this light, and not as admissions of prior art.

An electrocardiogram (“ECG”) graphs voltage acquired from a person's body over time. The voltages represent electrical activity of the heart. To acquire the voltages, electrodes are placed at selected points on the person's body. It is desirable to establish a strong physical contact and electromagnetic coupling between each of the electrodes and the person's body. The strong physical contact and electromagnetic coupling are desirable because they promote good data acquisition that improves the accuracy of the ECG.

One measure of the strength of the contact and the coupling is “contact impedance”, There inherently exists an impedance at the interface between the electrode and the skin and this impedance is called the contact impedance. A low contact impedance is desirable because it indicates a strong physical contact and electromagnetic coupling. Conversely, a high impedance is undesirable and may even indicate a “lead-off” condition. A lead-off condition is a condition in which the electrode has become detached from the person's body to the point it no longer adequately acquires the voltages.

ECG monitors may therefore monitor the contact impedance of the various electrodes during an ECG procedure. If a contact impedance exceeds some predetermined threshold, the ECG monitor may presume it indicates that a lead is off and issue an alarm. A clinician, upon detecting the alarm, may then check to make sure there are no detached electrodes and, if there are, then reattach them.

### BRIEF DESCRIPTION OF THE DRAWINGS

The present disclosure is best understood from the following detailed description when read with the accompanying figures, wherein:

FIG. 1 illustrates a patient monitoring system according to one or more examples;

FIG. 2 is a perspective view of an ECG lead set for use with the patient monitoring system of FIG. 1;

FIG. 3 is an illustration of a portion of an ECG signal representing a human heartbeat;

FIGS. 4A, 4B and 4C are a flow diagram of a method for ECG lead set on/off detection and type detection according to one or more examples;

FIG. 5 is a flow diagram of a method of operating a patient monitoring system according to one or more examples; and

FIG. 6 is a block diagram of a computing resource implementing a method of operating a patient monitoring system according to one or more examples.

It is emphasized that, in accordance with the standard practice in the industry, various features are not drawn to scale. In fact, the dimensions of the various features may be arbitrarily increased or reduced for clarity of discussion or illustration.

### DETAILED DESCRIPTION

Illustrative examples of the subject matter claimed below are disclosed. In the interest of clarity, not all features of an actual implementation are described for every example in this specification. It will be appreciated that in the development of any such actual implementation, numerous implementation-specific decisions may be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

The expressions such as “include” and “may include” which may be used in the present disclosure denote the presence of the disclosed functions, operations, and constituent elements, and do not limit the presence of one or more additional functions, operations, and constituent elements. In the present disclosure, terms such as “include” and/or “have”, may be construed to denote a certain characteristic, number, operation, constituent element, component or a combination thereof, but should not be construed to exclude the existence of or a possibility of the addition of one or more other characteristics, numbers, operations, constituent elements, components or combinations thereof.

As used herein, the article “a” is intended to have its ordinary meaning in the patent arts, namely “one or more.” Herein, the term “about” when applied to a value generally means within the tolerance range of the equipment used to produce the value, or in some examples, means plus or minus 10%, or plus or minus 5%, or plus or minus 1%, unless otherwise expressly specified. Further, herein the term “substantially” as used herein means a majority, or almost all, or all, or an amount with a range of about 51% to about 100%, for example. Moreover, examples herein are intended to be illustrative only and are presented for discussion purposes and not by way of limitation.

As used herein, to “provide” an item means to have possession of and/or control over the item. This may include, for example, forming (or assembling) some or all of the item from its constituent materials and/or, obtaining possession of and/or control over an already-formed item.

Unless otherwise defined, all terms including technical and/or scientific terms used herein have the same meaning as commonly understood by one of ordinary skill in the art to which the present disclosure pertains. In addition, unless otherwise defined, all terms defined in generally used dictionaries may not be overly interpreted. In the following, details are set forth to provide a more thorough explanation of the embodiments. However, it will be apparent to those skilled in the art that embodiments may be practiced without these specific details. In other instances, well-known structures and devices are shown in block diagram form or in a schematic view rather than in detail in order to avoid obscuring the embodiments. In addition, features of the different embodiments described hereinafter may be combined with each other, unless specifically noted otherwise. For example, variations or modifications described with respect to one of the embodiments may also be applicable to other embodiments unless noted to the contrary.

Further, equivalent or like elements or elements with equivalent or like functionality are denoted in the following description with equivalent or like reference numerals. As the same or functionally equivalent elements are given the same reference numbers in the figures, a repeated description for elements provided with the same reference numbers may be omitted. Hence, descriptions provided for elements having the same or like reference numbers are mutually exchangeable.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

Directional terminology, such as “top”, “bottom”, “below”, “above”, “front”, “behind”, “back”, “leading”, “trailing”, etc., may be used with reference to the orientation of the figures being described. Because parts of embodiments can be positioned in a number of different orientations, the directional terminology is used for purposes of illustration. It is to be understood that other embodiments may be utilized and structural or logical changes may be made without departing from the scope defined by the claims. The following detailed description, therefore, is not to be taken in a limiting sense. Directional terminology used in the claims may aid in defining one element's spatial or positional relation to another element or feature, without being limited to a specific orientation.

Instructions may be executed by one or more processors, such as one or more central processing units (“CPU”), digital signal processors (“DSPs)”, general purpose microprocessors, application specific integrated circuits (“ASICs”), field programmable logic arrays (“FPGAs”), or other equivalent integrated or discrete logic circuitry. Accordingly, the term “processor,” as used herein refers to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. A “controller,” including one or more processors, may use electrical signals and digital algorithms to perform its receptive, analytic, and control functions, which may further include corrective functions. Thus, a controller is a specific type of processing circuitry, comprising one or more processors and memory, that implements control functions by way of generating control signals.

A sensor refers to a component which converts a physical quantity to be measured to an electric signal, for example, a current signal or a voltage signal. The physical quantity may for example comprise electromagnetic radiation (e.g., photons of infrared or visible light), a magnetic field, an electric field, a pressure, a force, a temperature, a current, or a voltage, but is not limited thereto.

Signal conditioning, as used herein, refers to manipulating an analog signal in such a way that the signal meets the requirements of a next stage for further processing. Signal conditioning may include converting from analog to digital (e.g., via an analog-to-digital converter), amplification, filtering, converting, biasing, range matching, isolation and any other processes required to make a sensor output suitable for processing after conditioning.

As used herein, the term “smart” cable refers to cables that include embedded circuitry that is configured to operate to some extent interactively and autonomously with another device. A smart cable may include an electrically erasable programmable read-only memory (EEPROM), a central processing unit (CPU), a digital signal processor (DSP), a general-purpose microprocessor, an application specific integrated circuit (ASIC), a field programmable logic array (FPGA), or other equivalent integrated or discrete logic circuitry. In contrast, a “legacy” cable is one that that does not include embedded circuitry that is configured to operate to some extent interactively and autonomously with another device.

Smart cables include smart ECG lead sets and legacy cables include legacy ECG lead sets. ECG lead sets typically comprise one or more conductive leads having a connector at one end for connecting to an ECG monitor (patient monitor), and an electrode or electrode connector at another end for facilitating an electrical connection to a patient. In addition to enabling the transfer of data between a patient monitor and a smart ECG lead set, a smart ECG lead set may also have the capability of being automatically detectable by a patient monitor. In other words, a patient monitor may automatically detect that a smart lead set has been connected.

The subject matter described herein is directed to examples of patient monitoring systems having cable type detection capabilities and connection status testing capabilities. In various examples herein, the cable type detection and connection status testing may be implemented in a manner which minimizes interference with ECG signals carried on ECG leads coupled to a patient.

In some ECG monitoring systems, a patient monitor may detect patient cable connection status, e.g., whether a patent cable is plugged or unplugged from a monitor, by communicating with an EEPROM device built into an ECG lead set. This communication may be performed based on the industry-standard 1-Wire Bus protocol. When a patient monitor seeks to determine ECG lead set connection status and ECG lead set type identification, a lack of response may indicate that the cable is disconnected. When a response is received at the patient monitor, the cable identification (“cable ID”) within the response, such as via the 1-Wire Bus protocol, is used to identify the ECG set type. A patient monitor may further perform electrode impedance detection to ascertain the quality of connection of ECG electrodes to the patient.

A number of ECG lead set types are available, including lead sets including varying numbers of cable leads, such as, for example, 6LD (six leads), 5LD (five leads), 3LD (three leads), and so on. An ECG monitor may perform cable connection status and cable type identification on a periodic basis, for example, once or twice per second, such that any cable plug event or unplug event may be promptly captured. Once a cable is determined to be plugged into the monitor, cable type detection may be performed to ascertain how many electrodes the cable is intended to support.

It is to be noted that in the field of electrocardiography, and herein, a distinction is made between “ECG leads” and “conductive leads,” also referred to herein as “cables.” A patient monitor system may include an ECG lead set having some number of conductive leads (cables), e.g., a particular number of conductive cables or wires having an electrode at one end adapted to be attached to a patient. The number of ECG leads for a given ECG lead set does not necessarily equate to the number of conductive leads, since an EGG lead may be derived from a combination of electrical signals on two or more conductive leads. For example, four conductive leads (right arm, right leg, left arm, and left leg), may provide six ECG leads (right arm, left arm, left leg, augmented voltage left (aVL), augmented voltage right (aVR), and augmented voltage feet (aVF)).

In some cases, the periodic inquiries from a patient monitor to an ECG lead set may induce noise into the ECG signals transmitted from the patient to the monitor via the conductive leads. In examples herein, a system is provided to detect cable connection status and cable type detection while minimizing noise in the ECG signals.

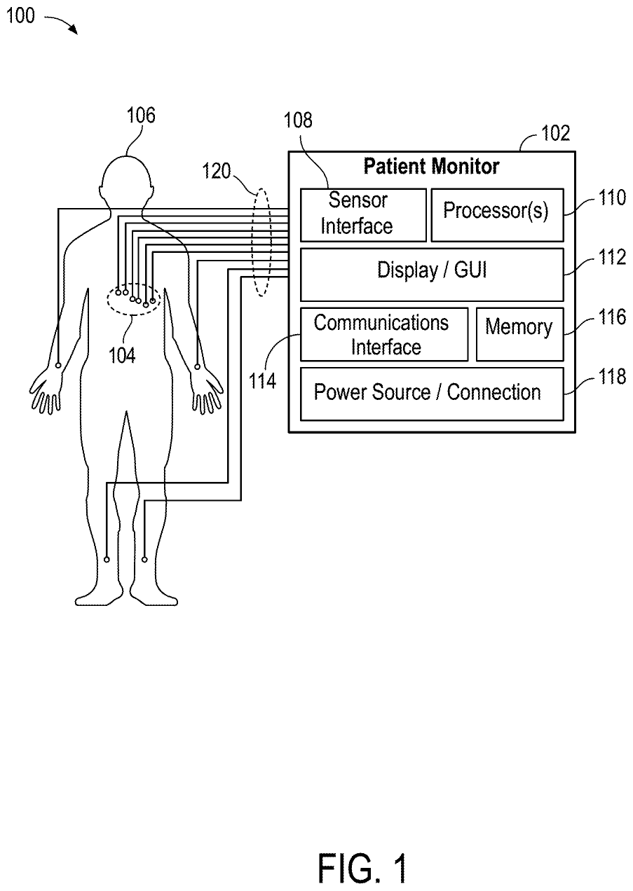

FIG. 1 shows a physiological monitoring system 100 according to one or more examples. As shown in FIG. 1, the system includes a patient monitor 102 (e.g., a physiological monitoring device) capable of receiving physiological data from various sensors 104 connected to a patient 106. In this example, sensors 104 comprise ECG electrodes affixed to the skin of patient 106.

In general, it is contemplated by the present disclosure that patient monitor 102 includes electronic components and/or electronic computing devices operable to receive, transmit, process, store, and/or manage patient data and information associated performing the functions of the system, which encompasses any suitable processing device adapted to perform computing tasks consistent with the execution of computer-readable instructions stored in a memory or a computer-readable recording medium.

Further, any, all, or some of the computing devices in patient monitor 102 may be adapted to execute any operating system, including Linux®, UNIX®, Windows Server®, etc., as well as virtual machines adapted to virtualize execution of a particular operating system, including customized and proprietary operating systems. Patient monitor 102 may be further equipped with components to facilitate communication with other computing devices over one or more network connections, which may include connections to local and wide area networks, wireless and wired networks, public and private networks, and any other communication network enabling communication in the system.

As shown in FIG. 1, patient monitor 102 may be, for example, a patient monitor implemented to monitor various physiological parameters of patient 106 via sensors 104. Patient monitor 102 may include a sensor interface 108, one or more processors 110, a display/graphical user interface (GUI) 112, a communications interface 114, a memory 116, and a power source (or power connection) 118. Sensor interface 108 may be implemented in hardware or combination of hardware and software and is used to connect via wired and/or wireless connections to sensors 104 for gathering physiological data from the patient 106. As noted, sensors 104 in the present example are ECG electrodes affixed to the skin of patient 106. A plurality of conductive leads 120, comprising a plurality of conductive cables, are provided for coupling sensors 104 to sensor interface 108. In one or more examples, conductive leads 120 comprise a plurality of ECG cables. In other embodiments, connections 102 may comprise one or more wired or wireless communication channels configured to at least transmit sensor data from sensors 104 to sensor interface 108.

The data signals from the sensors 104 may include, for example, sensor data related to an ECG. The one or more processors 110 may be used for controlling the general operations of patient monitor 102, as well as processing sensor data received by sensor interface 108. The one or more processors 110 may be, but are not limited to, a central processing unit (“CPU”), a hardware microprocessor, a multi-core processor, a single core processor, a field programmable gate array (“FPGA”), a microcontroller, an application specific integrated circuit (“ASIC”), a digital signal processor (“DSP”), or other similar processing device capable of executing any type of instructions, algorithms, or software for controlling the operation and performing the functions of patient monitor 102.

Display/GUI 112 may be configured to display various patient data, sensor data, and hospital or patient care information, and includes a user interface implemented for allowing interaction and communication between a user and patient monitor 102. Display/GUI 112 may include a keyboard (not shown) and/or pointing or tracking device (not shown), as well as a display, such as a liquid crystal display (“LCD”), cathode ray tube (“CRT”) display, thin film transistor (“TFT”) display, light-emitting diode (“LED”) display, high definition (“HD”) display, or other similar display device that may include touch screen capabilities. Display/GUI 112 may provide a means for inputting instructions or information directly to the patient monitor 102. The patient information displayed may, for example, relate to the measured physiological parameters of patient 106 (e.g., ECG readings).

Communications interface 114 may enable patient monitor 102 to directly or indirectly (via, for example, a monitor mount) communicate with one or more computing networks and devices, workstations, consoles, computers, monitoring equipment, alert systems, and/or mobile devices (e.g., a mobile phone, tablet, or other hand-held display device). Communications interface 114 may include various network cards, interfaces, communication channels, cloud, antennas, and/or circuitry to enable wired and wireless communications with such computing networks and devices. Communications interface 114 may be used to implement, for example, a Bluetooth® connection, a cellular network connection, and/or a WiFi® connection with such computing networks and devices. Example wireless communication connections implemented using the communication interface 6 include wireless connections that operate in accordance with, but are not limited to, IEEE802.11 protocol, a Radio Frequency For Consumer Electronics (RF4CE) protocol, and/or IEEE802.15.4 protocol (e.g., ZigBee® protocol). In essence, any wireless communication protocol may be used.

Additionally, communications interface 114 may enable direct (i.e., device-to-device) communications (e.g., messaging, signal exchange, etc.) such as from a monitor mount to patient monitor 102 using, for example, a universal serial bus (“USB”) connection or other communication protocol interface. The communication interface 6 may also enable direct device-to-device connection to other devices such as to a tablet, computer, or similar electronic device; or to an external storage device or memory.

Memory 116 may be a single memory device or one or more memory devices at one or more memory locations that may include, without limitation, one or more of a random-access memory (“RAM”), a memory buffer, a hard drive, a database, an erasable programmable read only memory (“EPROM”), an electrically erasable programmable read only memory (“EEPROM”), a read only memory (“ROM”), a flash memory, hard disk, various layers of memory hierarchy, or any other non-transitory computer readable medium. Memory 116 may be used to store any type of instructions and patient data associated with algorithms, processes, or operations for controlling the general functions and operations of patient monitor 102.

Power source/connection 118 may include a self-contained power source such as a battery pack and/or include an interface to be powered through an electrical outlet (either directly or by way of a monitor mount). Power source 118 may also be a rechargeable battery that can be detached allowing for replacement. In the case of a rechargeable battery, a small built-in back-up battery (or super capacitor) can be provided for continuous power to be provided to patient monitor 102 during battery replacement. Communication between the components of patient monitor 102 in this example (may be established using an internal bus (not explicitly shown in FIG. 1).

Patient monitor 102 may be attached to one or more of several different types of sensors 104 and may be configured to measure and readout physiological data related to patient 106. As noted, sensors 104 may be attached to patient monitor 102 by conductive leads 120 which may be, for example, cables coupled to sensor interface 108. Additionally, or alternatively, one or more sensors 104 may connected to sensor interface 108 via a wireless connection. In which case sensor interface 108 may include circuitry for receiving data from and sending data to one or more devices using, for example, a WiFi® connection, a cellular network connection, and/or a Bluetooth® connection.

The data signals received from sensors 104, may be analog signals. For example, the data signals for the ECG may be input to sensor interface 108, which can include an ECG data acquisition circuit (not shown separately in FIG. 1). An ECG data acquisition circuit may include amplifying and filtering circuitry as well as analog-to-digital (A/D) circuitry that converts the analog signal to a digital signal using amplification, filtering, and A/D conversion methods. In the event that the ECG sensor is a wireless sensor, sensor interface 108 may receive the data signals from a wireless communication module. Thus, sensor interface 108 is a component which may be configured to interface with one or more sensors 104 and receive sensor data therefrom.

As further described herein, the processing performed by an ECG data acquisition circuit may generate analog data waveforms or digital data waveforms that are analyzed by, in this particular embodiment, a microcontroller. However, other embodiments may use other kinds of processors. The microcontroller may be one of the processors 110.

The microcontroller, for example, analyzes the digital waveforms to identify certain digital waveform characteristics and threshold levels indicative of conditions (abnormal and normal) of the patient 106 using one or more monitoring methods. A monitoring method may include comparing an analog or a digital waveform characteristic or an analog or digital value to one or more threshold values and generating a comparison result based thereon. The microcontroller may be, for example, a processor, an FPGA, an ASIC, a DSP, a microcontroller, or similar processing device. The microcontroller may include a memory or use a separate memory 116. The memory may be, for example, a RAM, a memory buffer, a hard drive, a database, an EPROM, an EEPROM, a ROM, a flash memory, a hard disk, or any other non-transitory computer readable medium.

Memory 116 may store software or algorithms with executable instructions and the microcontroller may execute a set of instructions of the software or algorithms in association with executing different operations and functions of patient monitor 102 such as analyzing the digital data waveforms related to the data signals from sensors 104.

As noted, in the example of FIG. 1, conductive leads 120 between sensors 104 and sensor interface 108 may be an ECG lead set. In this example, conductive leads 120 may include a plurality of leads with each lead terminating at a sensor 104 that is attached to the patient for measuring ECG data. It is noted that in practice, an ECG lead set 120 may include any number of conductive leads.

As noted above, a “smart” ECG lead set may include circuitry to provide additional functionality compared to “legacy” lead sets (as detailed hereinbelow). For example, a smart ECG lead set may be configured to operate to some extent interactively and autonomously with patient monitor 102.

A smart ECG lead set may include circuitry (such as an EEPROM or other semiconductor chip) that is automatically activated when it detects that it has been plugged in and receives power from a patient monitor via one or more of the conductive leads of the ECG lead set. The circuitry may use EEPROM to automatically identify itself to patient monitor 102, which may include transmitting authentication information, lead information (e.g., indicting a number of leads). Patient monitor 102 may automatically detect the smart ECG lead set when electrical contact is made and activate its ECG front-end circuitry to enable patient monitor 102 to read and process ECG measurements.

An EEPROM in a smart ECG lead set may be configured to store data relevant to the ECG lead set, including information such as a number of leads, authentication information, number-of-use information, and so forth.

The number of conductive leads of various types of ECG lead sets can vary. For example, existing ECG lead sets may have anywhere between three to ten (or more) conductive leads. However, any number of leads is possible. Patient monitor 102 may process information on as many conductive leads as are present in the ECG front end. This processing allows for proper processing and display of ECG data by patient monitor 102. For example, the ECG lead set may implement an EEPROM (or other processor) to enable the monitor to communicate with, and read information from, the EEPROM. On the other hand, when a legacy ECG cable (e.g., without an EEPROM) is used, patient monitor 102 may not automatically obtain information regarding the number of leads.

As noted, an EEPROM in a smart ECG lead set may store usage information indicating its number of uses of the lead set and provide the number of uses to patient monitor 102 so that patient monitor 102 may determine whether the ECG lead set has exceeded a specified number of uses. For example, some ECG lead sets are manufactured for a one-time use and may not have been designed or manufactured to be repeatedly sanitized and reused. For another example, a one-time use ECG lead may experience degradation of materials over time from repeated exposure to chemicals, to which the ECG leads may not be intended to be exposed. Alternatively, one-time use ECG leads may have hard-to-clean surface or components in which bacteria or other germs could grow and affect subsequent patients upon repeated use. A smart ECG lead set may keep track of its number of uses by incrementing a counter and storing the number of uses. Alternatively, patient monitor 102 may track the number of uses and write the number of uses into the EEPROM of a smart ECG lead set. Thus, patient monitor 102 may be capable of carrying out both read and write operations with respect to EEPROM data in a smart ECG lead set. A smart ECG lead set may also store information that identifies the lead set as one with electrosurgical unit (ESU) protection or without ESU protection.

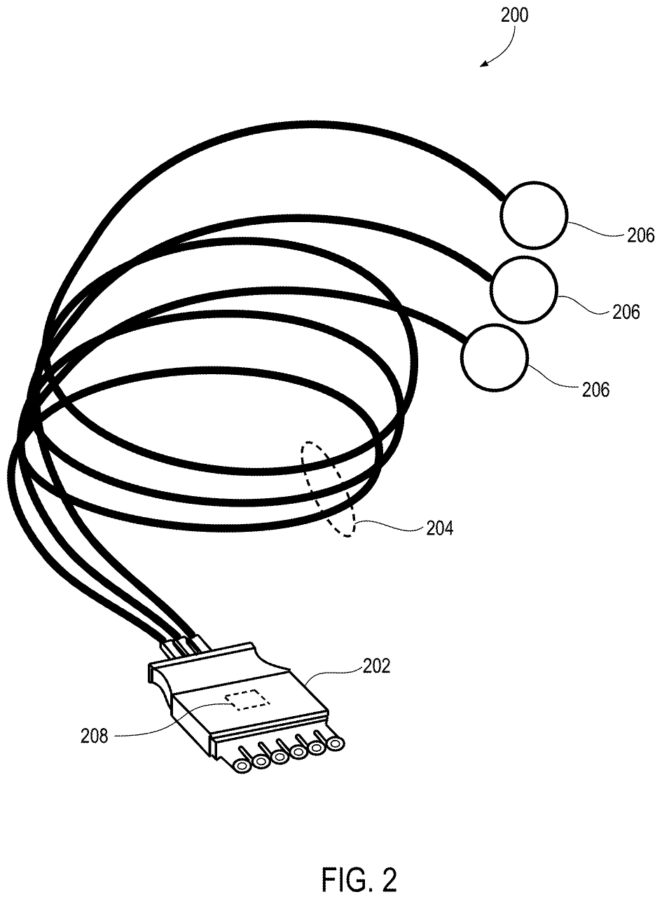

FIG. 2 is a perspective illustration of an example ECG lead set 200 which may serve as the connections 120 in the patient monitory system 100 of FIG. 1 between a patient (106 in FIG. 1) and patient monitor 102. In the example of FIG. 2, ECG lead set 200 includes a connector 202 for connecting to sensor interface 108 of patient monitor 102 of FIG. 1. ECG lead set 200 of FIG. 2 further includes a plurality of connections 204 comprising a plurality of cables. Lead set 200 of FIG. 2 further includes a plurality of electrode connectors 206 for connection to patient electrodes (not shown) affixed to the skin of patient 106.

In the example of FIG. 2, a three-lead (3LD) ECG electrode set is depicted, including three conductive leads. As noted, however, ECG electrode sets may have any number of conductive leads, most commonly between three and ten.

As shown in FIG. 2, ECG lead set 200 is a smart lead set including an EEPROM 208 (or other circuitry, such as an electrically-programmable read-only memory (EPROM), an application-specific integrated circuit (ASIC), or other custom integrated circuitry) for communicating with patient monitor 102. In various examples, communication between EEPROM 208 in ECG lead set 200 and patient monitor 102 may be performed using the aforementioned 1-Wire Bus protocol. This may enable patient monitor 102 to ascertain the presence of ECG lead set 200 (e.g., that ECG lead set 200 is physically connected to patient monitor 102), as well as the type of ECG lead set 200 (e.g., the number of leads that ECG lead set 200 has, and/or whether ECG lead set 200 includes an electro-surgical (“ESU”) block suitable for high-current operating room conditions). ECG lead set 200 may include a separate conductive wire (not shown) for enabling communication via the 1-Wire Bus protocol. However, in some examples, such a separate conductive wire may not be electrically shielded from the other conductive leads, leading to the possibility of electrical interference with ECG signals conducted through the ECG lead set 200.

In one or more examples, patient monitor 102 may interrogate ECG lead set 200 periodically, for example, once every one-half or one second, so that any lead set plug/unplug event may be detected. Once a lead set such as lead set 200 from FIG. 2 has been plugged in and detected (e.g., connection status determination), through communication with EEPROM 208, patient monitor may then ascertain how many electrodes the lead set is capable of supporting (e.g., type determination). Thereafter, a lead on/off detection algorithm executed, for example by processor(s) 110 in patient monitor 102, can continue to periodically monitor ECG lead set connection status.

As noted, however, in some cases, such as when using the 1-Wire Bus communication protocol, the periodic interrogation of ECG lead set 200 by patient monitor 102 may induce noise into the electrical ECG signals communicated by connections 204 from patient 106 to patient monitor 102. This may adversely affect the ability of patient monitor to process and display the ECG readings of patient 106.

Therefore, and according to one or more examples herein, a system is provided to minimize interference with ECG signals due to ECG lead set connection status determination and type determination. In one or more examples, rather than performing interrogations of an ECG lead set at strictly defined periodic intervals, the interrogations may be performed in synchronization with detected ECG signals, in order that communications between patient monitor 102 and an ECG lead set such as ECG lead set 200 cause minimal interference with detected ECG signals at patient monitor 102.

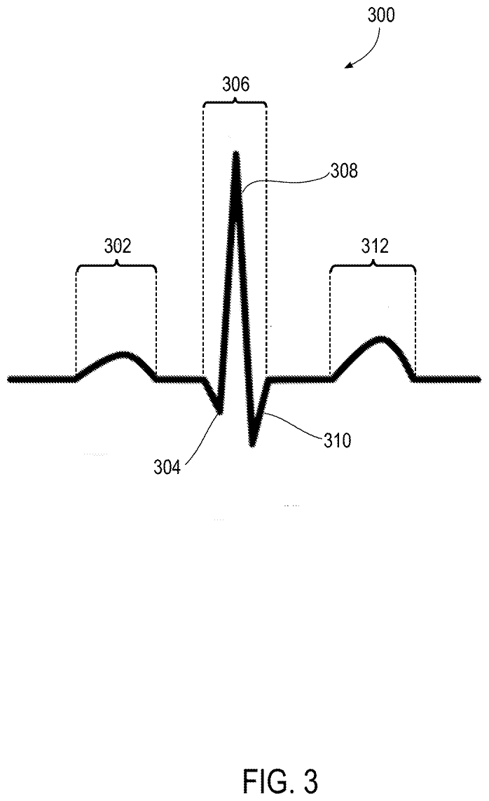

FIG. 3 is a portion of a plot of an ECG trace 300 from a single, typical, cardiac cycle, e.g., a single heartbeat. An ECG trace includes several distinct intervals and features. At the beginning of a cardiac cycle, there is a “P-wave,” denoted by reference numeral 302 in FIG. 3 and generally corresponding to atrial contraction/depolarization. The “Q-wave,” denoted by reference numeral 304 in FIG. 3 signals the onset of ventricular contraction/depolarization. Ventricular contraction/depolarization occurs during the “QRS complex,” denoted by reference numeral 306 in FIG. 3 and consisting of Q-wave 304, an “R-wave” denoted by reference numeral 308 and an “S-wave” denoted by reference numeral 310. A “T-wave” denoted by reference numeral 312 occurs after QRS complex 306 and corresponds to repolarization/repolarization of the ventricles in advance of the next heartbeat. In a typical cardiac rhythm, there is a period of time between the T-wave of a first cardiac cycle and the P-wave of the next cardiac cycle during which the ECG trace is relatively flat (e.g., there is relatively little cardiac electrical activity to be sensed). In examples herein, a patient monitor may take advantage of these periods of decreased cardiac electrical activity to conduct connection status and type determination interrogations with minimal interference with ECG signals.

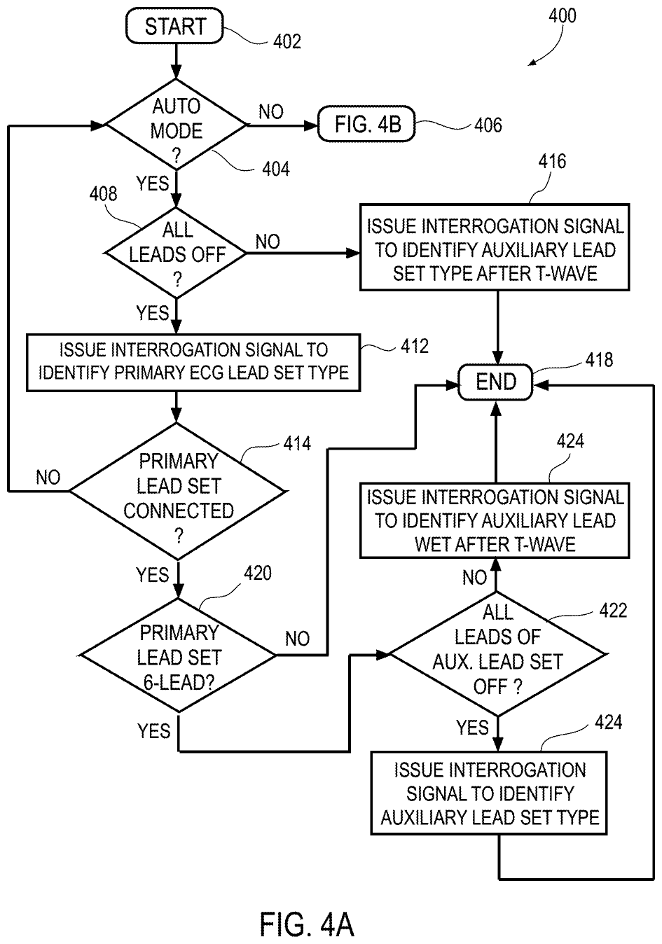

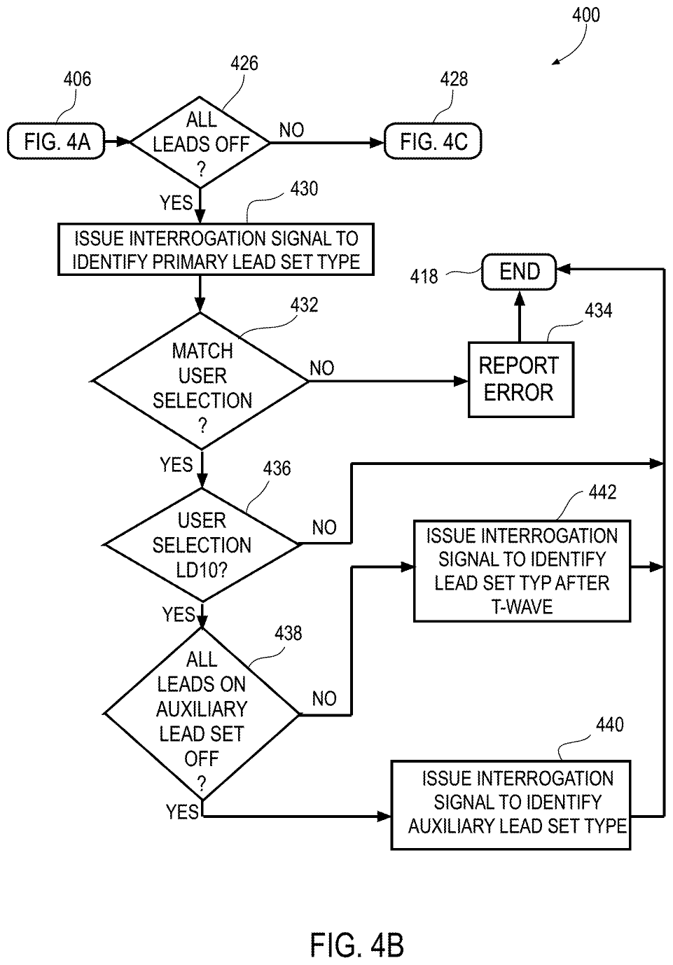

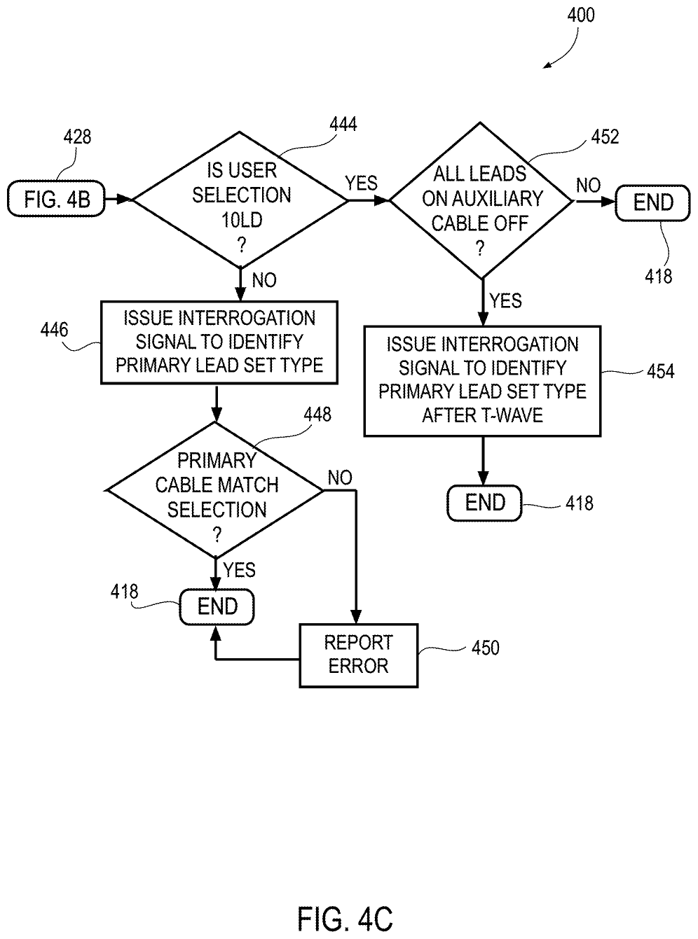

FIGS. 4A, 4B, and 4C together are a flow diagram illustrating a method 400 for minimizing interference in ECG signals due to connection status and type determination in a patient monitoring system. With reference first to FIG. 4A, the method 400 according to one or more examples starts at block 402. Block 402 represents the issuance of an interrogation to an ECG lead set, which, as noted above, may initially be programmed in patient monitor 102 to occur periodically, for example, once every one-half or one second. According to one or more examples, however, and as described hereinbelow, the exact timing of each scheduled ECG lead set interrogation issued by patient monitor 102 may be modulated (e.g., delayed) according to sensed ECG signals, in order to minimize interference with sensed ECG signals by the interrogation signals.

In decision block 404, a determination is made in patient monitor 102 whether a clinician has placed patient monitor 102 into an “auto” mode, in which patient monitor 102 is to automatically determine the type of ECG lead set is connected, as opposed to a “manual” mode, in which a clinician may manually designate an ECG lead set type, such as by interacting with display/GUI 112. FIG. 4A represents an example process for operation of patent monitor system 100 when operated in “auto” mode, while FIG. 4B represents an example process for operation of patient monitor system 100 when operated in “manual” mode. Thus, at decision block 404 in FIG. 4A, if “auto” mode has not been selected, operational flow proceeds to what is depicted in FIG. 4B, as shown by block 406 in FIG. 4A.

With continued reference to FIG. 4A, however, if “auto” mode has been selected, in decision block 408 a determination is made whether all leads of an ECG lead set are “off.” This determination may be made by, for example, measuring impedance at each ECG lead set input to sensor interface 108.

If, in block 408, it is determined that all leads are off, then in block 412 patient monitor 102 issues an interrogation signal, via a 1-Wire bus connection, for example, to determine the primary ECG lead set type that is connected to patient monitor 102. Issuance of an interrogation signal may involve, for example, sending one or more packets of data across the communications path (e.g., the 1-Wire bus connection) and waiting and/or receiving a response. In response to that interrogation signal, if it is determined that no primary cable has been connected or detected, then from decision block 412 flow returns to block 402.

Returning to decision block 408, if it is determined that not all of the leads are off (e.g., that impedance readings reflect connection of an ECG lead set), then flow proceeds to block 416, wherein patient monitor 102 waits until after occurrence of a T-wave in a detected ECG signal to interrogate to determine what type of auxiliary cable type has been installed. An auxiliary cable may be required for any ECG lead set which requires more than six physical connections to a patient monitor. By waiting until after occurrence of a T-wave to perform the interrogation in block 416, interference with ECG signals may be minimized. Following this interrogation, the process ends, at block 418, to be resumed upon a next interrogation cycle as herein described. In examples, the next interrogation cycle may be programmed to occur in one-half or one second following the previous interrogation cycle, but, as described herein, and according to one or more examples herein, may be delayed in order to synchronize with detected ECG signals, as herein described.

On the other hand, if at decision block 414 a primary cable type has been detected, flow proceeds to decision block 420, wherein a determination is made whether the primary ECG lead set is a 6-lead ECG lead set. If not, then the process ends, again at block 418, to be resumed upon a next cycle as herein described.

If at decision block 420 the primary ECG lead set is not a six-lead ECG block, flow proceeds to block 422, wherein a determination is made whether all leads of an auxiliary cable are off. Again, this determination may be made, for example, through impedance measurements for an auxiliary cable connection. If auxiliary cable connections are not off at block 422, then, in block 424, patient monitor 102 will wait until after occurrence of a T-wave in a detected ECG signal to interrogate to determine the type of auxiliary cable attached. Again, by waiting until after occurrence of a T-wave, interference with sensed ECG signals is minimized. Following this interrogation, the process flow ends, again at block 418

On the other hand, at decision block 420, if a determination is made that the primary ECG lead set is a six-lead ECG lead set, then flow proceeds selectively to block 422 where again a determination is made whether all leads of the auxiliary cable are off, and if so, flow proceeds to block 424, wherein patient monitor 102 may immediately (e.g., without waiting until after occurrence of a T-wave in the sensed ECG signal) interrogate to determine the type of the auxiliary ECG lead set that is attached. Following this interrogation, the process flow ends, again at block 418.

Turning now to FIG. 4B, the portion of process flow 400 for patient monitoring system 100 in the event that a manual mode of operation has been selected is depicted, as established at decision block 404 in FIG. 4A. As shown in FIG. 4B, connecting to FIG. 4A at block 406, in decision block 426, patient monitor 102 determines whether all leads of an ECG lead set are off, again, such as through impedance measurements at sensor interface 108. If not, then, as shown in FIG. 4C, connecting to FIG. 4B at block 428, in decision block 444, patient monitor 102 determines whether the use has selected a ten-lead (10LD) configuration. If not, in block 446, patient monitor 102 issues an interrogation signal to identify the primary lead type. Then, in decision block 448, patient monitor 102 determines whether the primary lead set type identified in block 446 matches the user selection. If so, the processor flow ends, again at block 118; if not, in block 450, an error is reported and the process flow ends at block 418,

On the other hand, if patient monitor 102 determines in decision block 44 that the user has selected a ten-lead configuration, then in decision block 452 patient monitor 102 determines whether all leads on a primary cable are off. If not, process flow ends at block 418, If not, patient monitor 102 issues an interrogation signal, in block 454 to identify the primary lead set type following a T-wave, after which the process flow ends in block 118.

Returning to FIG. 4B, at block 426, if all leads are determined to be off, then in block 430, patient monitor 102 may interrogate the ECG lead set to determine its type. In decision block 432, patient monitor 102 determines whether the ECG lead set determined in block 430 matches the ECG lead set type specified by the clinician in manual mode. If not, then in block 434, an error may be reported, such as on display/GUI 112, to alert the clinician of the discrepancy.

If, in decision block 432, the ECG lead set type determined in block 430 matches that specified by the clinician in manual mode, then in decision block 436, a determination is made whether the clinician selected a ten-lead ECG lead set. If not, the process comes to an end at block 418. If a ten-lead ECG lead set has been selected, then, in block 438, a determination is made whether all of the leads of an auxiliary ECG lead set are off. If all leads are off, then in block 440, patient monitor 102 may interrogate the auxiliary ECG lead set to determine its type. On the other hand, if all leads of the auxiliary lead set are not off in block 438, then, in block 442, patient monitor 102 may wait until after occurrence of a T-wave in detected ECG signals to interrogate the auxiliary ECG lead set to determine its type. Once again, by waiting until after a T-wave, interference with ECG signal detection may be minimized. Following this interrogation in block 440, the process ends, at block 418.

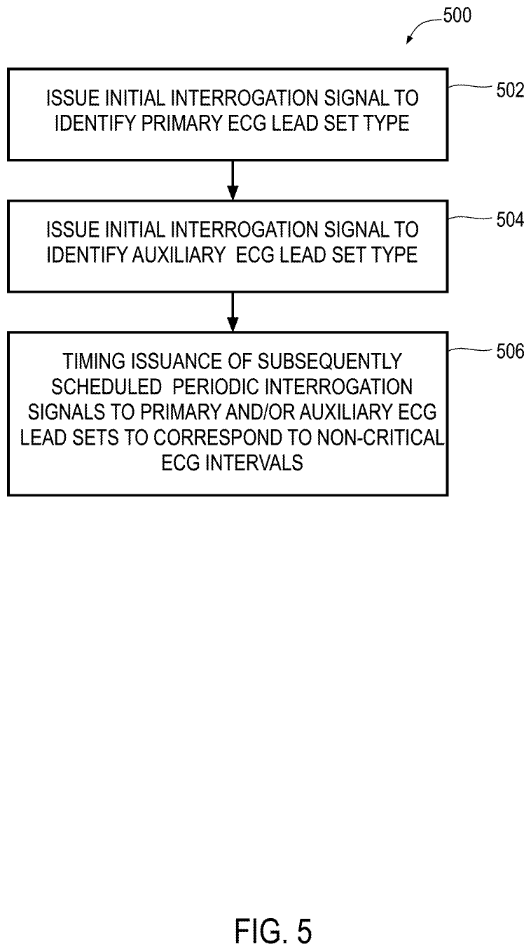

FIG. 5 is a flow diagram summarizing a method 500 of operating of a patient monitoring system such as patient monitoring system 100 of FIG. 1 according to one or more examples. As shown in block 502 of FIG. 5, patient monitor 102 may issue an initial interrogation signal to identify a primary ECG lead set type, and in block 504, patient monitor 102 may issue an initial interrogation signal to identify any auxiliary ECG lead set type.

Subsequently, in block 506, patient monitor 102 may time issuance of subsequently scheduled period interrogation signals to the primary and/or secondary ECG lead sets to correspond to non-critical ECG intervals. As used herein, the term non-critical ECG intervals refers to intervals in which the ECG signal from the patient is either unreliable or unavailable. An example of a non-critical ECG intervals is reflected in the operation described herein with reference to FIGS. 4A and 4B, wherein following initial interrogations, patient monitor 102 delays issuance of interrogation signals until after detection of T-waves in the patient ECG signals. As noted above, the interval following a T-wave in a cardiac cycle preceding a subsequent P-wave is non-critical since very little ECG activity is typically occurring during such times.

Other examples of non-critical ECG intervals include, without limitation, periods of time during which patient monitor 102 is adjusting its signal processing algorithms. For example, based on impedance measurements and ECG readings, patient monitor 102 may periodically adjust its signal processing, such as by re-designation of a particular ECG lead as a reference (ground) lead, or accommodating the detection of a loss of signal on a lead of an otherwise connected ECG lead set. Another example is when patient monitor 102 detects excessive voltage(s) on one or more ECG leads, in which case patient monitor may enter an “auto-blocking” mode which limits (i.e., artificially attenuates) the ECG signals on an ECG lead. Yet another example is so-called Wilson Point updating, during which patient monitor 102 may re-define the combinations of leads used to generate a displayed ECG signal. Still another example of a non-critical ECG interval is when patient monitor 102 detects, such as through impedance measurements, that connection of one or more leads to a patient has been compromised.

In all of these non-critical intervals, the ECG signal is either in transition, unreliable, or unavailable. Thus, during these non-critical intervals, any interference that might be introduced by issuance of ECG lead set interrogation signals will be inconsequential. By taking advantage of the occurrence of such non-critical ECG intervals, interference with more meaningful ECG data may be minimized.

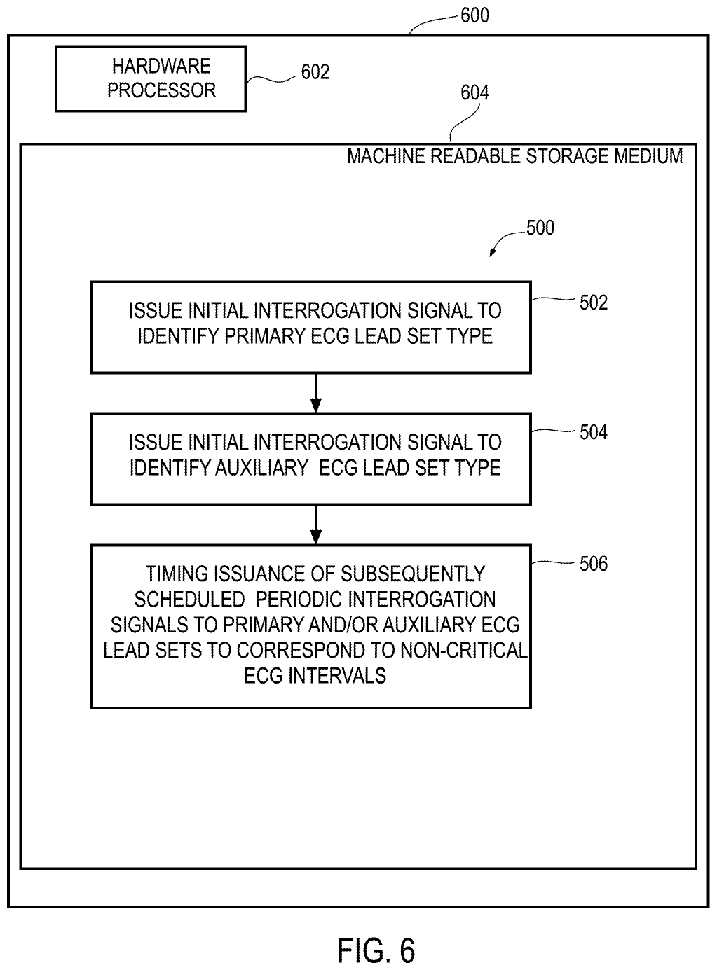

FIG. 6 is a block diagram representing a computing resource 600 implementing a method of controlling a cooling fan in a compute device according to one or more examples. Computing resource 600 may include at least one hardware processor 602 and a non-transitory machine-readable storage medium 604. As illustrated, machine readable medium 604 may store instructions, that when executed by hardware processor 602 (either directly or via emulation/virtualization), cause hardware processor 602 to perform the method 500 described above with reference to FIG. 5.

In various examples, hardware processor 602 may be, for example and without limitation, a microcontroller, a central processing unit (“CPU”), a digital signal processor (“DSP”), a programmed logic array (“PLA”), or a custom processing circuit.

A computer-readable media may be any available media that may be accessed by a computer. By way of example, such computer-readable media may comprise random access memory (“RAM”), read-only memory (“ROM”), electrically-erasable/programmable read-only memory (“EEPROM”), compact disc ROM (“CD-ROM”) or other optical disk storage, magnetic disk storage or other magnetic storage devices, or any other medium that may be used to carry or store desired program code in the form of instructions or data structures and that may be accessed by a computer. Disk and disc, as used herein, includes compact disc (“CD”), laser disc, optical disc, digital versatile disc (“DVD”), floppy disk and Blu-ray® disc where disks usually reproduce data magnetically, while discs reproduce data optically with lasers.

Note also that the software implemented aspects of the subject matter hereof are usually encoded on some form of program storage medium or implemented over some type of transmission medium. The program storage medium is a non-transitory medium and may be magnetic (e.g., a floppy disk or a hard drive) or optical (e.g., a compact disk read only memory, or “CD ROM”), and may be read only or random access. Similarly, the transmission medium may be twisted wire pairs, coaxial cable, optical fiber, or some other suitable transmission medium known to the art. The claimed subject matter is not limited by these aspects of any given implementation.

The foregoing description, for purposes of explanation, uses specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. Examples herein are not intended to be exhaustive of or to limit this disclosure to the precise forms described. Many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

What is claimed is:

1. A method of operating a patient monitoring system, comprising:
issuing a first initial interrogation signal from a patient monitor to identify a primary electrocardiogram (“ECG”) lead set type coupled to the patient monitor;
selectively issuing a second initial interrogation signal from the patient monitor, depending upon the identified type of the primary ECG lead set to identify an auxiliary ECG lead set; and
timing issuance of at least one subsequent interrogation signal to the primary ECG lead set to correspond to a non-critical ECG interval.

2. The method of claim 1, wherein the non-critical ECG interval comprises an interval following occurrence of a T-wave in a detected ECG signal and prior to occurrence of a subsequent P-wave in the detected ECG signal.

3. The method of claim 1, wherein the non-critical EGG interval comprises an interval in which the patient monitor is limiting a voltage on an ECG lead of the primary EGG lead set or the secondary ECG lead set.

4. The method of claim 1, wherein the non-critical ECG interval comprises an interval in which the patient monitor is adjusting ECG signal processing parameters.

5. The method of claim 1, wherein the initial interrogation signal and at least one subsequent interrogation signal are communicated to circuitry included in the primary ECG lead set.

6. A patient monitoring system, comprising:
a patient monitor having a processor and a user interface for displaying an electrocardiogram (“ECG”) signal; and
a primary ECG lead set, coupled to the patient monitor, for providing ECG signals to the patient monitor;
wherein:
the patient monitor issues an initial interrogation signal to identify the type of the primary ECG lead set;
the patient monitor times issuance of at least one subsequent interrogation signal to the ECG lead set to correspond to a non-critical ECG interval.

7. The patient monitoring system of claim 6, wherein the non-critical ECG interval comprises an interval following occurrence of a T-wave in a detected ECG signal and prior to occurrence of a subsequent P-wave in the detected ECG signal.

8. The patient monitoring system of claim 6, wherein the non-critical ECG interval comprises an interval in which the patient monitor is limiting a voltage on an ECG lead of the ECG lead set.

9. The patient monitoring system of claim 6, wherein the non-critical ECG interval comprises an interval in which the patient monitor is adjusting ECG signal processing parameters.

10. The patient monitoring system of claim 6, wherein the initial interrogation signal and at least one subsequent interrogation signal are communicated to circuitry included in the primary ECG lead set.

11. A non-transitory computer-readable medium, tangibly embodying instructions executable by a hardware processor to:
issue an initial interrogation signal from a patient monitor to identify a primary electrocardiogram (“ECG”) lead set type coupled to the patient monitor;
selectively issue an initial interrogation signal from the patient monitor, depending upon the identified type of the primary ECG lead set, to identify an auxiliary ECG lead set; and
time issuance of at least one subsequent interrogation signal to the primary ECG lead set to correspond to a non-critical ECG interval.

12. The non-transitory computer-readable medium of claim 11, wherein the non-critical ECG interval comprises an interval following occurrence of a T-wave in a detected ECG signal and prior to occurrence of a subsequent P-wave in the detected ECG signal.

13. The non-transitory computer-readable medium of claim 11, wherein the non-critical ECG interval comprises an interval in which the patient monitor is limiting a voltage on an ECG lead of the ECG lead set.

14. The non-transitory computer-readable medium of claim 11, wherein the non-critical ECG interval comprises an interval in which the patient monitor is adjusting ECG signal processing parameters.
